(19) United States
(12) Patent Application Publication
(10) Pub. No.: US 2021/0000674 A1
(43) Pub. Date: Jan. 7, 2021

(54) MECHANICAL DEVICE FOR TEMPOROMANDIBULAR JOINT DISTRACTION

(71) Applicants: INSTITUTO POLITECNICO DE COIMBRA, Coimbra (PT); UNIVERSIDADE DE COIMBRA, Coimbra (PT)

(72) Inventors: Julio Andre RAMALHO DA FONSECA, Cantahede (PT); Pedro Miguel GOMES NICOLAU, Figueira Da Foz (PT); Joao Paulo CARVALHO OLIVEIRA, TABUA (PT); Luis Manuel FERREIRA ROSEIRO, Coimbra (PT)

(21) Appl. No.: 16/977,541

(22) PCT Filed: Mar. 1, 2019
(86) PCT No.: PCT/IB2019/051669

§ 371 Date: Sep. 2, 2020

(30) Foreign Application Priority Data
Mar. 2, 2018 (PT) 110605

(51) Int. Cl. A61H 1/02

(52) U.S. Cl. CPC A61H 1/02; A61H 2201/1207; A61H 2201/1253; A61H 2201/1604; A61H 2201/5097; A61H 2201/1685; A61H 2201/169; A61H 2201/5007; A61H 2201/5061; A61H 2201/1676

(57) ABSTRACT

The present invention discloses a mechanical device for joint distraction of the Temporomandibular Joint (TMJ) comprising an upper arm (19) and lower arm (20) joined in a pivotal zone by a mechanical and pivoting component (5) and by a spring (6) or mechanical component between the arms (19) (20). Each arm has a handle (1) of the upper arm (19) and a handle (4) of the lower arm (20). Each arm has an intraoral mouth end for contact with dental surfaces: the upper arm (19) has a tray (2) with a flat support surface for the maxillary teeth flanked by a containment flap (11) of the upper tray; the lower arm (20) has a curvature until its end, with a customized surface (3) for supporting the lower teeth. It presents a mechanical component (13) that limits the extent of movement and force applied by the patient or clinician.

### FIELD OF THE INVENTION

Temporomandibular Disorders (TMD) are considered a heterogeneous group of psychophysiological disorders of the stomatognathic system.^{[1-2]} They cover a wide spectrum of muscular, skeletal (or both) clinical problems.^{[3-6]} They often start with pain, joint sounds and limited function/mandibular movement, being considered one of the main causal factors of non-dental orofacial pain.^{[7]} Conservative and non-invasive treatment is considered as the first choice at an early stage, since the symptomatology is usually reduced through the combined use of occlusal devices, physical therapy and medication.^{[8]}

Temporomandibular Joint (TMJ) joint disc displacements are the most common arthropathy and are characterized by several stages of clinical dysfunction involving an abnormal interrelationship of the disc condyle complex (more often an anterior or anteromedial disc displacement).^{[9]} Pain (in acute cases), changes in mandibular movement pattern and joint noises are the most frequent symptoms. The causes of disc displacements are not completely established. It is postulated that, in the majority of cases, the elongation or rupture of the condyle-disc ligaments allow the disc displacement.^{[10]} Changes in the lubrication and quality of synovial fluid have also been suggested as possible etiological agents.^{[11]} The presence of osteoarthritis may also precipitate changes in the condyle-disc complex.^{[8]}

In addition to the dentist intervention with occlusal devices (among other resources), mandibular physical therapy aims to reduce musculoskeletal pain, promote muscle relaxation, reduce muscle hyperactivity, improve muscle control and function, and maximize joint mobility. In addition to the electro-therapeutic means, there are several manual therapy techniques directed to the TMJ that aim the joint decompression, fibrosis and adhesions reduction in structures such as ligaments or the joint capsule, reduction of the articular disc displacement or adaptation of the retrodiscal tissues. These are intra oral techniques, such as distraction of TMJ or specific exercises of joint mobility or muscle strengthening.^{[12]}

In the specific case of TMJ disc displacements, the TMJ distraction technique is one of the most common used therapeutic resources. It is a technique that aims to increase the space between the mandibular condyle and the temporal bone articular fossa, decompressing the joint and promoting the adaptation of the articular tissues and/or the disc recapture. It is a technique almost exclusively performed by the physical therapist and/or dentist, with repeated applications over an extended period of treatment, which may reach several weeks or months, and difficult to perform manually by the patient in an ambulatory basis.^{[8]}

Currently there are only ambulatory mandibular exercise devices that aim to increase the range of mandibular movement through rotation, rototranslation and condylar translation. Thus, the creation of a device capable of produce or assist patients in the distraction maneuver of the TMJ will be a valuable aid in the treatment of these pathologies, increasing patient adherence and reducing costs related to a long treatment time. In this context, the main aim of the present invention is the development of a biomechanical device, with innovative features, named “TMJ Distractor—A Physical Therapy Auxiliary of Patients with Temporomandibular Disorders”. This device allows the TMJ distraction technique to be implemented by both the physical therapist and/or the dentist and by the patient as well, under medical indication. The device of this invention is composed by a set of easily assembled mechanical components which allow customization for each patient. This device has an original design, allowing the upper arch to be stabilized with an upper tray and having a customizable end in the lower arm. The resulting movement at the TMJ level will increase the space between the condyle and the mandibular fossa. The chin can be easily stabilized by the patient with one hand, while the other hand operates the device. It is an easy-to-use and patient-friendly device with user-friendly features. The versatility of the device allows its use in both the right and left joints. The adjustable mechanical safety system ensures that the proper force and displacement for each patient are not exceeded.
[1] Okeson J P. Fundamentals of occlusion and temporomandibular disorders. St. Louis: Mosby; 1985.
[2] Okeson J P. Management of temporomandibular disorders and occlusion. 3rd ed. St. Louis: Mosby-Year Book; 1993.
[3] Rossetti L M, Rossetti P H, Conti P C, de Araujo Cdos R. Association between sleep bruxism and temporomandibular disorders: a polysomnographic pilot study. Cranio 2008; 26(1):16-24.
[4] Wadhwa S, Kapila S. TMJ disorders: future innovations in diagnostics and therapeutics. J Dent Educ 2008; 72(8):930-47.
[5] Makino M, Masaki C, Tomoeda K, et al. The relationship between sleep bruxism behavior and salivary stress biomarker level. Int J Prosthodont 2009; 22(1):43-8.
[6] Nagamatsu-Sakaguchi C, Minakuchi H, Clark G T, Kuboki T. Relationship between the frequency of sleep bruxism and the prevalence of signs and symptoms of temporomandibular disorders in an adolescent population. Int J Prosthodont 2008; 21(4):292-8.
[7] Magnusson GECT. Management of Temporomandibular Disorders in the General Dental Practice: Quintessence Publishing Co; 1999.
[8] De Leeuw R, American Academy of Orofacial. Pain., American Academy of Orofacial Pain. Orofacial pains guidelines for assessment, diagnosis, and management. 4th ed. Chicago: Quintessence; 2008.
[9] Isberg-Holm A M, Westesson P L. Movement of disc and condyle in temporomandibular joints with clicking. An arthrographic and cineradiographic study on autopsy specimens. Acta Odontol Scand 1982; 40(3):151-64.
[10] Stegenga B, de Bont L G, Boering G, van Willigen J D. Tissue responses to degenerative changes in the temporomandibular joint: a review. J Oral Maxillofac Surg 1991; 49(10):1079-88.
[11] Nitzan D W. The process of lubrication impairment and its involvement in temporomandibular joint disc displacement: a theoretical concept. J Oral Maxillofac Surg 2001; 59(1):36-45.
[12] Craane B, Dijkstra P U, Stappaerts K, De Laat A. Randomized controlled trial on physical therapy for TMJ closed lock. J Dent Res 2012; 91(4):364-9.

### BACKGROUND OF THE INVENTION

A research of the existing devices was done and it was verified that the currently existing ambulatory mandibular exercise devices intend only to increase the range of mandibular movement through rotation, rototranslation and/or condylar translation, not allowing the implementation of the distraction technique of the TMJ. The distraction technique of the TMJ differs markedly from the other ones, since it aims to increase the vertical space between the mandibular condyle and the articular temporal fossa, decompressing the joint and promoting the adaptation of the articular tissues and or the disc recapture, while the anterior ones were essentially concerned with range of mouth opening, and may even be counterproductive in cases of joint disc displacement.

Three devices are highlighted in this research: a) the OraStretch Press Jaw Motion Rehab System (unknown Patent—device marketed by TheraBite Corporation); b) TheraBite System (U.S. Pat. No. 5,035,420A); c) TheraPacer Jaw CPM (U.S. Pat. No. 4,883,046 A). These devices are produced in synthetic polymeric material, easy to clean by the patients themselves. None of these devices distracts the TMJ, they only seek to meet the need to increase the mandibular range of motion, essentially using the condylar rotation movement. Its ends are supported on the anterior teeth of both arches with a consequent produced movement of mouth opening in rotation or rototranslation. They do not produce the TMJ distraction movement in any case, as they do not create a vertical separation movement of the temporal fossa and condyle, achieved by the present invention due to the exclusive support of its lower end in the most distal tooth of the lower arch. The previous devices may be indicated for cases of post-surgical rehabilitation considering the joint mobilization needs of these patients. However, in cases of disc displacement, these devices may even be counterproductive. Thus, based on the researches carried out, it can be affirmed that no registered patents were found that allow to evidence the existence of any system that performs, globally, the objective functions and claims of this invention. In view of the above, the device whose protection in the form of a patent application is requested assumes characteristics of true innovation, without direct competition with the same therapeutic indications.

### BRIEF DESCRIPTION OF THE DRAWINGS

FIG. 1—Schematic and simplified perspective representation, according to the invention; upper view. It illustrates the relationship between the upper (19) and lower (20) arms of the device, as well as the angulation/curvature of its ends.

FIG. 2—Schematic and simplified perspective representation, according to the invention; exploded view of the device. It illustrates the relationship between the various components of the invention.

FIG. 3—Schematic and simplified perspective representation, according to the invention; bottom exploded view of the device. It illustrates the relationship between the various components of the invention.

FIG. 4—Schematic and simplified perspective representation, according to the invention; lateral view, with the device assembled, handles (1) and (4) apart and ends (3) and (2) closed/approximated. It illustrates the relationship between the upper (19) and lower (20) arms of the device.

FIG. 5—Schematic and simplified perspective representation, according to the invention; assembled device view, handles (1) and (4) apart and ends (3) and (2) closed/approximated. It shows the relationship between the upper arm (19) and lower arm (20) of the device, namely between the upper tray (2) for jaw engagement and the lower end (3).

FIG. 6—Schematic and simplified perspective view, according to the invention, of the upper arm (19); upper view. Illustrates the shape of the upper tray (2) for jaw engagement.

FIG. 7—Schematic and simplified perspective view, according to the invention, of the upper arm (19); side view.

FIG. 8—Schematic and simplified perspective representation, according to the invention; anterior-superior view of the upper arm (19) of the device.

FIG. 9—Schematic and simplified perspective representation, according to the invention; top view of the lower arm (20) of the device. Illustrates the end with customizable surface (3) for supporting the lower teeth.

FIG. 10—Schematic and simplified perspective representation, according to the invention; side view of the lower arm (20) of the device. It illustrates the handle (4) of the lower arm (20) of the device, perforated, and the engaging surface (16) for the removable piece (15) for the mandibular incisors.

FIG. 11—Schematic and simplified perspective representation, according to the invention; view of the lower arm (20) of the device. Illustrates the end with a customizable surface (3) for supporting the lower teeth and the notch surface (10) for engaging the spring (6) or mechanical component between the upper arm (19) and the lower arm (20).

### DETAILED DESCRIPTION OF THE INVENTION

With reference to the figures and their subtitling, the characterization of the mechanical device of the present invention is further described in detail. This device (TMJ distractor) corresponds to a physical therapy auxiliary for patients with articular TMJ disorders. Its design guarantees the application of the TMJ distraction technique, a practice that aims to increase the vertical space between the mandibular condyle and the articular fossa of the temporal bone, decompressing the joint and promoting the adaptation of the articular tissues and/or the disc recapture.

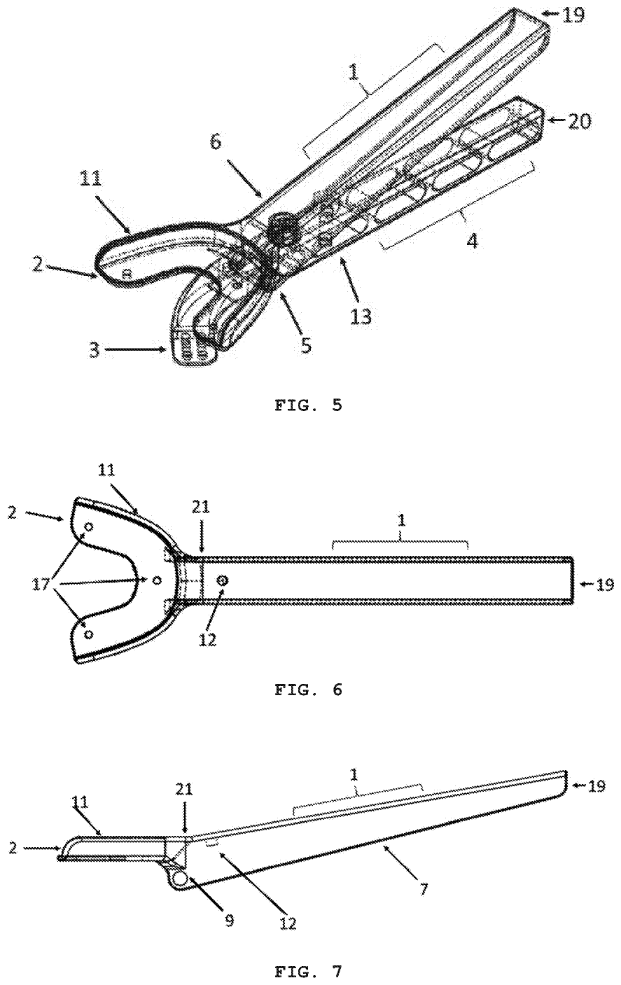

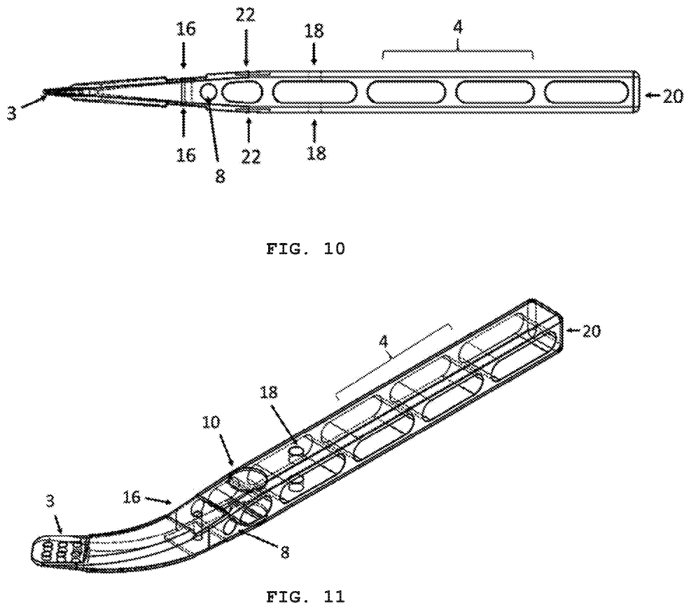

The present invention is based on a mechanical configuration having two main structural elements: a top structural member, designated upper arm (19) shown in FIGS. 6, 7 and 8, and a lower structural member, designated lower arm (20), shown in FIGS. 9, 10 and 11. These two components, (19) and (20), are connected and articulate in a hinge zone by a mechanical and pivoting component (5). This mechanical and pivoting component (5) is inserted through a hole (9) of the upper arm (19) and a hole (8) of the lower arm (20) for fixation of the upper arm (19) to the lower arm (20). The holes (8) and (9) may have an adaptable shape to the type of mechanical component (5) considered for articulation between the arms.

The mechanical component (5) allows an inter-arms rotation movement relatively to its axis it may be a cylindrical pin or spindle having a length which fills the interior space, with an internal thread at its two ends, where it tightens a screw for its locking and stabilization with respect to the two arms. This component may be made of a metallic material, in particular stainless steel, or plastic material, as nylon or teflon.

Each arm has a handle and an end. By the movement of the handles around the mechanical and pivoting component (5), that is, by approximating the handle (1) of the upper arm (19) of the device to the handle (4) of the lower arm (20) of the device, is produced an inter-arms pivotal movement with separation of the arms ends (2) and (3) with consequent production of the distraction movement of the TMJ. The ends of the arms will be inserted intraorally into close contact with the maxillary teeth and the mandibular target teeth. With this positioning and support of the device, a mechanical lever effect will be introduced during its inter-arm movement with fulcrum in the zone of the maxillary teeth contact with the upper tray (2), and that it will introduce a downward force into the lower molars in contact with the customizable surface (3) for supporting the lower teeth. This force will have a perpendicular direction to the mandibular teeth surface plane and the overall result of the TMJ movement will correspond to the increase of distance between the articular surfaces of the condyle and the articular fossa of the temporal bone, with the consequent TMJ distraction movement. A secondary chin support point, with manual (operator or patient) application of a vertical upward force, can maximize the desired effect and is therefore recommended.

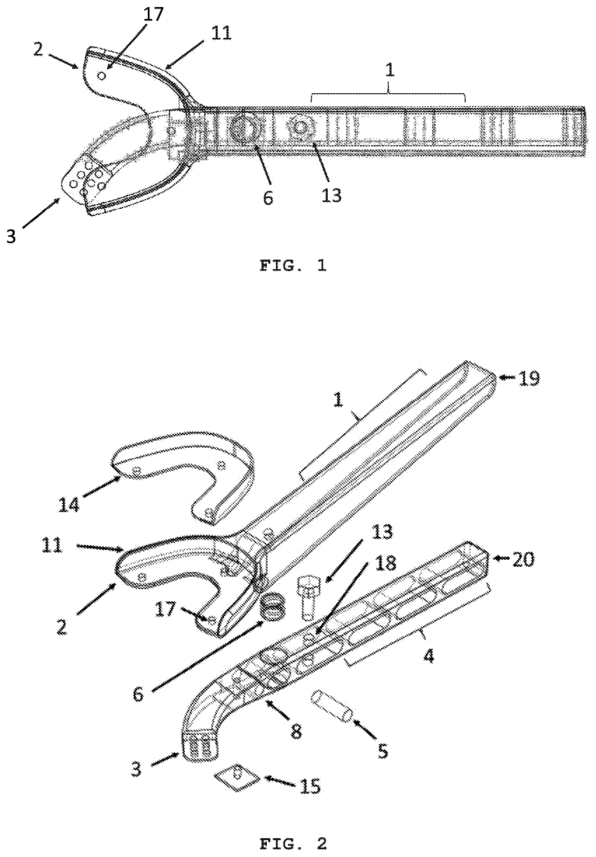

As it can be seen in FIGS. 1 to 8, the upper arm (19) has a handle (1) and its end corresponds to an upper tray (2) with a flat support surface for the maxillary teeth, as seen in FIGS. 5, 6 and 8, flanked by a containment flap (11) of the upper tray (2). The lower arm (20), as seen in the FIGS. 2, 3 and 9 to 11, has a handle (4) and its end has a curvature preceding a customizable surface (3) for supporting the lower teeth. The arms (19) and (20) may be constructed in one piece or in two pieces and, as a result, their ends may be fixed or separable from the handles, and may be easily exchanged, using locking or retaining mechanisms, or others adapted to that means, localized in the separation zone (21) between the handle (1) of the upper arm (19) and its end or tray (2) as well as in the separation zone (22) between the handle (41) of the lower arm and its end (3).

The, locking and retaining mechanism, effect is obtained with a male-female mechanical system, wherein the removable end has an external V-shaped protrusion and the remainder of the arm an internal protrusion, also in V-shape. These two protrusions are complementary and have a surface finishing that ensures the stability of the two parts of the arm by sliding and fitting mechanisms. In the case of the lower arm (20), this assembly is produced by sliding in the horizontal plane, and the lower end (3) can be moved, with the preceding curvature, being inserted from left to right or vice versa. In the case of the upper arm (19), the assembly is produced in the vertical plane, with the insertion of the removable part from top to bottom. The handle (1) of the upper arm (19) of the device has surface which can be smooth or rough, and can be ergonomically customizable, and a lower half cane (7) which allows the fitting of the handle (4) of the lower arm (20) of the device.

In its middle zone, in the transition from the handle (1) to the upper tray (2), the upper arm (19) presents the hole (9) for the mechanical and pivoting component (5) for articulation of the upper arm (19) to the lower arm (20). The upper tray (2) will have a smooth or rough surface for contact of the maxillary teeth. In addition, it may be customized, typically through elastomeric material, composite, acrylic resin or other adapted to that mean, ensuring the fitting through the retaining holes (17) of the tray, custom or customizable prosthetic elements, in the most diverse types of materials. This is represented by the removable tray (14) for maxillary teeth contact/engagement which fits into the retaining holes (17).

This tray (14) may be customizable or not, made in several materials (plastics, composites, acrylic resins or elastomers), ensuring the fitting of the upper dental arch of custom or customizable prosthetic elements, in the most diverse types of materials.

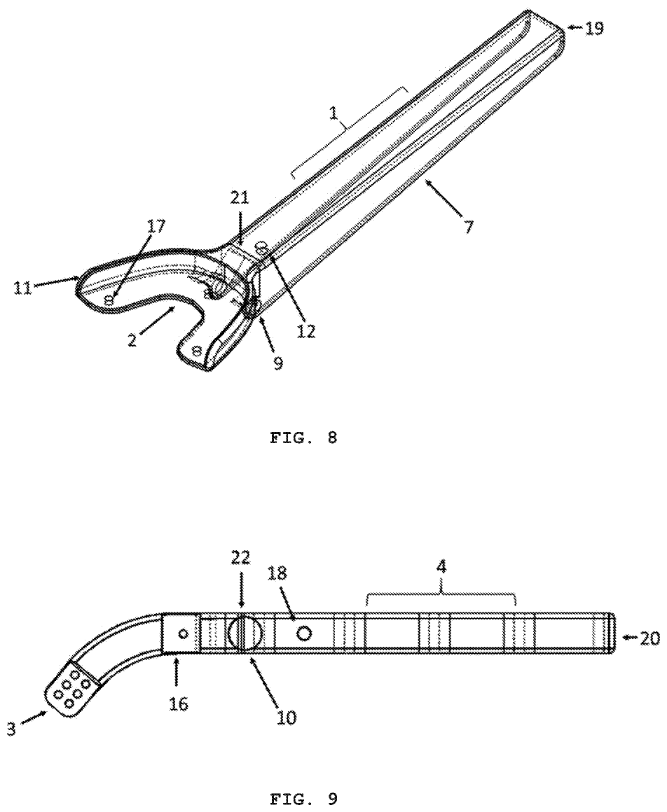

The upper tray (2) is flanked and laterally reinforced by a containment surface or containment flap (11) of the upper tray. This flap works as a lip and cheek protection. In addition, this flap (11) will also allow the fitting of a customizable secondary part with acrylic or any other material, or the fitting of trays, custom or customizable prosthetic elements in various materials. On the underside of the handle (1) of the upper arm (19) of the device there is an area (12) of engagement and retention of the spring (6) or mechanical component between the upper arm (19) and the lower arm (20), as shown in FIGS. 6 and 8. The spring (6) between the upper (19) and lower (20) arms engages in the notch surface (10) of the lower arm (20) and in the engagement area (12) of the upper arm (19).

This component (6) may be a compression spring or other material with equivalent elastic behavior (for example an elastomeric cylinder), with rigidity and elasticity suitable to ensure the return of the movement between the upper (19) and lower (20) arms, after the application of the force that approximates the handles (1) and (4) with the consequent spacing of the ends (3) and (2) with the production of the resultant distraction movement of the TMJ. Its rigidity and elasticity should be known and adjusted to the needs of the device and the user, with regard to the mechanical effect desired by the device, but also with regard to user safety issues.

This spring (6) may be replaced by an active mechanical part with ends adapted to the assembly and retention area (12) and to the notch surfaces (10). This mechanical component may comprise a micromotor or electromechanical programmable device, allowing the linear movement to be carried out on its axis and consequently separation or approach between the upper (19) and lower (20) arms. The active mechanical component must have programmable and adjustable characteristics by the clinician or user, having associated the considered necessary variables, such as range of motion, cycle time and frequency of movement. The lower arm of the device is formed by its handle (4) and by its end with a customizable surface (3) for supporting the lower teeth. The handle (4) of the lower arm (20) of the device has a smooth surface (can be rough or ergonomically customizable) and holes along its body for the main purpose of reducing its weight. These holes should have a geometry and position so as not to interfere with the mechanical performance of the device, ensuring adequate strength and stiffness of the arm. They may thus have different shapes and sizes from that shown in the drawing, but must provide the appropriate mechanical characteristics, in order to fit the components to be included.

All the distractor activity may be recorded through one or more electronic monitoring units. Among the monitoring possibilities, reference should be made in particular to the force applied by the operator hand (or digital) at the extremities of the arms, which can be quantified by the placement of a piezoresistive or piezoelectric sensor in the surfaces of the arms. Also, the number of cycles of use can be guaranteed by means of an inertial mass unit (IMU), which incorporates an accelerometer and a gyroscope. This type of sensors is normally very small, can be connected to the monitoring unit, also with small size, that can establish Bluetooth contact with a mobile device (PC, android platform or IOS platform). In the case of application of an active mechanical component in place of the spring it can also be connected to the monitoring unit.

This microelectronic monitoring unit, therefore small in size, may be optimized to ensure assembly in one of the handle (4) holes of the lower arm (20), thereby being coupled and protected in the process of using the distractor.

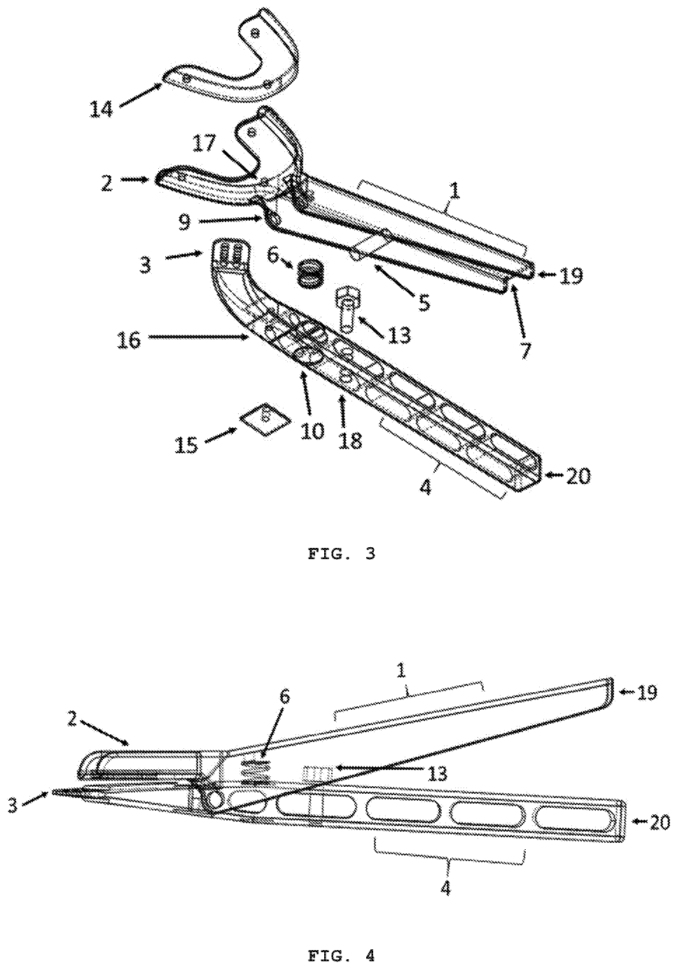

The handle (4) of the lower arm (20) has on both (upper and lower) faces a notch surface (10) for assembly of the spring (6) or mechanical component between the upper arm (19) and the lower arm (20), according to FIGS. 2, 3, 9 and 11. At its end the lower arm (20) of the device has a curvature up to the customizable surface (3) for supporting the lower teeth. This surface (3) can be customizable or not, have a smooth or rough surface to fit the mandibular teeth, can be customized with acrylic or any other material (elastomeric, composite or other), or allow the insertion of trays, custom or customizable prosthetic elements, in the most diverse materials. The material selected for customization, once retained in the holes of the customizable surface (3), can print the target teeth by placement and intra-oral adjustment. FIGS. 2 and 3 show that in its middle zone, the lower arm (20) of the device has a hole (6) for the mechanical component (5) for upper (19) and lower (20) arm assembly and articulation.

The lower arm (20) of the device has symmetry between its upper and lower faces. It can then be rotated around its longer axis to allow the left or right. TMJ to be treated, using only the same device and not two different devices, depending on the patient's indications or needs. This allows a greater versatility of the device, minimizing costs through a simple and rapid inversion of the part, and not a different part for each side to be treated. It also facilitates the use of the device by the professional and the user. This objective may be fulfilled in particular by simply rotating the lower arm of the device (the entire arm) or only its end (3) with the curvature that precedes it, in the case of being removable, as the ends of the handles may be detachable as previously mentioned.

Rotation of the entire lower arm (20) is achieved by the temporary removal of the mechanical component (5), removal of the mechanical component (13) to limit the extent of movement and force and disassembly of the spring (6) (or elastomer or active mechanical device). Following the separation of the upper arm (19) from the lower arm (20), the lower arm (20) can be rotated, and then mounted and positioned the mechanical component to limit the extent of movement and force (13), the spring (6) and the mechanical and pivoting component (5). The device will become a functional system again, but now with the symmetrical position of the lower arm (20). After its positioning, the plastic removable part (customizable) for the mandibular incisors (15) is disassembled and positioned on the opposing face on the engaging surface (16).

In case the rotation is implemented only with the removable end (3), which can be separable from the handle (4), this end is disassembled by exerting a horizontal shear force, in the separation zone (22) between the handle (4) and the end with a customizable surface (3), which allows the sliding of the V-shaped surfaces. After removal, this part is rotated along its longer axis, and its assembled, by pressing and sliding between the V-shaped contact surfaces. After its positioning, the removable plastic piece (customizable) for contact with the mandibular incisors (15) is disassembled and positioned on the opposite face of the removable end on the engaging surface (16).

As shown in the FIGS. 2, 3 and 11, the lower arm (20) of the device has a hole (18) on its two faces (upper and lower), for fixation of the mechanical component to limit the extent of movement and force (13). This mechanical component (13) is formed by a screw with a normal or special head, which will be positioned by adjustment in the orifice (18) and which will have a variable length depending on the distraction needs. The screw can be made of metallic or polymeric material, and it can integrate at its end several sensors, allowing the identification of important variables for an intelligent operation of the device, such as a contact sensor for circuit activation or a piezoresistive sensor for measuring the force of contact. In the case of their implementation, these sensors are embedded in the screw head and will be connected to the electronic monitoring unit. The contact sensor may trigger a light or sound signal to the distractor user with biofeedback purposes.

In the process of using the distractor, by the approximation of the upper arm (19) to the lower arm (20), when the lower surface of the upper arm touches the head of the screw (13), movement is blocked, acting as a mechanical movement end and it is not possible to introduce more movement and/or force into the teeth and consequently into the TMJ, even if there is an increase of force at the extremities of the arms, by manual tightening of the device.

This mechanical component (13) to limit the extension of movement and force will be fixed by screwing into the hole (18). The threads of the mechanical component (13) and of the hole (18) will be in fine pitch thread, with adequate precision and adjustment, in order to avoid becoming loose during the distractor use. As an alternative to the head screw, this mechanical component (13) may be a locking pin, in metallic or polymeric material, with the same characteristics of use. In this case, the adjustment will not be made on site, the component must have a brake previously adjusted to its length, suitable for a proper use. Its positioning in the hole (18) will be made by pressure, and the brake will act as a stop on the surface of the hole (18), so as to ensure that it does not changes the position during use of the distractor by sliding into the hole.

As seen in the FIGS. 2, 3 and 11, the lower arm (20) of the device has on its two faces an engaging surface (16) for a removable piece (15) for contact with the mandibular incisors. This piece (15) has a standard geometry, for fitting into the orifice and side edges, which will keep it stable, and may be constructed of elastomer, an acrylic resin or a suitable plastic. Due to the geometric characteristics of both the removable piece (15) and the engaging surface (16), this piece can be easily removed. The geometry of the engaging surface (16) allows, as an alternative to the removable piece (15), the fitting of trays, custom or customizable prosthetic elements, in various types of materials suitable for that purpose, which will make a more precise contact with the mandibular incisors.

The upper (1) and lower (4) handles of the device may have a different design, surfaces or geometry depending on the required ergonomics of the handle surfaces, and this ergonomics can be adjusted for manual or digital gripping, with either the left or right hand. Thus, the surface of the handles can be smooth, rough or individualized with wavy or rounded shapes. On the surface of the handles (1) and (4) a hand force (during the manipulation of the device) measurement sensor can be mounted, as previously mentioned. This sensor may be of the piezoresistive or piezoelectric type and may be connected to the electronic control unit embedded in one of the handle as described above. Alternatively, this sensor can operate autonomously, sending data via Bluetooth to a device for data acquisition and viewing (PC, Android or IOS).

In the case of use of only a force sensor at the end of the surface of the handles (1) and (4), as well as in the case of an integrated use with other sensors (electromyographic or temperature sensors, clocks, repetition sensors) control and/or biofeedback mechanisms can be implemented in the use of the device, both directed to clinicians responsible for treatment orientation and to the patient. In particular, the force sensor at the end of the handles (1) and (4) can be used autonomously and only in the process of adjusting the force limiter (13) and then removed, thus ensuring the highest effectiveness, safety and personal protection of the patient in its use. The components described above, as well as others that can be coupled to the device, will be able to have diverse sources of energy to guarantee its operation; may be connected to computer data control or monitoring systems, via physical or other connection that allows the transmission of information and may emit sound, light or other signals with an equivalent effect, both for the user and for the clinicians responsible for the treatment orientation. The device may be constructed integrally in one single material or in a combination of various materials it may be composed of metal alloy or polymeric material, produced by additive manufacturing technologies or subtractive manufacturing, as well as by casting or material injection.

1. Mechanical device for temporomandibular joint distraction, characterized in that it comprises:
a) an upper arm (19) and a lower arm (20) articulated in a pivoting zone by a mechanical component for articulation and pivoting (5) and by a spring (6) or mechanical component between the upper arm (19) and the lower arm (20);
b) each arm having a handle: handle (1) of the upper arm (19) of the device and the handle (4) of the lower arm (20) of the device;
c) each arm having an intraoral mouth end for contact with dental surfaces, the upper arm (19) having an upper tray (2) with a support surface for maxillary teeth and the lower arm (20) with a curvature up to its end, with a customizable surface (3) for supporting target lower teeth;
d) a mechanical component (5) for articulation and pivoting of the upper arm (19) to the lower (20) which permits a pivotal mechanism between arms;
e) a mechanical component (13) limiting the extent of movement and force.

2. Mechanical device according to claim 1 where in the two arms (19) and (20) are movable, in which by the movement of the handles (1) and (4) around the mechanical component articulation and pivoting (5), that is to say, by approximating the handle (1) of the upper arm (19) of the device to the handle (4) of the lower arm (20) of the device, an inter-arm movement separates the ends (3) and (2), which will produce a vertical downward force on the lower molars in contact with the customizable surface (3) for supporting the lower teeth with consequent production of the TMJ distraction movement.

3. Mechanical device according to claim 1 where in it comprises two arms in which:
a) the arms (19) and (20) may be built in one piece or in two pieces and consequently their ends may be fixed or separable from the handles and may be exchanged for others quickly by using locking or retaining mechanisms localized in the separation zone (21) between the handle (1) of the upper arm (19) and its upper end or tray (2) as well as in the separation zone (22) between the handle (4) of the lower arm (20)) and its end (3);
b) the locking and retaining mechanism in the regions (21) and (22) is obtained by a mechanical male-female type system (wherein the removable end has a V-shaped external protrusion and the remainder of the arm a internal protrusion, also in V-shape) and whose complementary geometry guarantees the stability of the two parts of the arms by sliding and fitting;
c) in the case of the lower arm (20), the assembly in the separation zone (22) between the handle (4) of the lower arm (20) and its end (3) is produced by sliding in the horizontal plane, with the removable lower arm end (3) with the preceding curvature being inserted from left to right or vice versa;
d) in the case of the upper arm (19), the assembly in the separation zone (21) between the handle (1) of the upper arm (19) and its end or tray (2) is produced in the vertical plane, with the insertion of the removable part (2) from top to bottom;
e) on the surface or body/holes of the handles (1) and (4) of the arms (19) and (20), hand-held force measurement sensors may be coupled, piezoresistive, piezoelectric or other type, which can be connected to electronic control units embedded in one of the handle holes, or alternatively, these sensors can operate autonomously, sending data via Bluetooth to a display and data acquisition device.

4. Mechanical device according to claim 1 wherein it comprises an upper arm (19) in which:
a) at its underside of the handle (1) of the upper arm (19) has an area (12) of assembly and retention of the spring (6) or mechanical component between upper and lower arms;
b) in the underside of the handle (1) has a lower half-cane (7) which ensures the fitting of the handle (4) of the lower arm (20);
c) in the middle zone of the upper arm (19), in the transition from the handle (1) to the upper tray (2), has a hole (9) in the upper arm (19) for the mechanical and pivoting component (5) for articulation between the upper arm (19) and the lower arm (20).

5. Mechanical device according to claim 1 wherein the upper arm (19) an upper tray-shaped end (2) with a support surface for the maxillary teeth in which:
a) the upper tray (2) may have a smooth or rough surface to support the maxillary teeth; may be customizable with elastomeric material, composite, acrylic resin or other; it may allow the insertion of elements, made by various types of materials through the retaining orifices (17);
b) the upper tray (2) may allow the assembly of a removable tray (14) for contact with the maxillary teeth (14) which assembles in the holes (17) for fitting the tray (14);
c) the upper tray (2) is flanked and laterally reinforced by a containment surface or containment flap (11) of the upper tray, which functions as a lip and cheek protection, and which may have various shapes or designs to allow the fitting of a customizable secondary piece with acrylic or any other material, or to allow the fitting of trays, custom or customizable prosthetic elements (or not), in the most diverse materials.

6. Mechanical device according to claim 5 wherein the removable tray (14) which can be customizable or not, made in a variety of materials, ensuring the fitting of the upper dental arch, custom or customizable prosthetic elements, in the different types of materials.

7. Mechanical device according to claim 1 wherein it comprises a lower arm (20) with a curvature up to its end (3), with a customizable surface for supporting the target lower teeth in which:
a) the surface (3) may be customizable or not, have a smooth or rough surface to fit the mandibular teeth, may be customizable with acrylic or any other material (elastomeric, composite or other), or allow the fitting of trays, custom or customizabled (or not), in the most diverse materials;
b) the material chosen for the customization of the surface (3), once retained in the holes of its surface can print the target teeth by placement and intra-oral adjustment.

8. Mechanical device according to claim 7 wherein the lower arm (20) which has a symmetry of design between its upper and lower faces.

9. Mechanical device according to claim 7 wherein it comprises a lower arm (20) in which:
a) the rotation along the longer horizontal axis of the entire lower arm (20) or only its end (3) allows alternating the treatment of the left or right TMJ, using only the same device and not two different devices, depending on the indications or needs of the user;
b) in the case of rotation of the entire lower arm (20) this is achieved by the temporary removal of the mechanical and pivoting component (5), removal of the mechanical component to limit the extent of movement and force (13), disassembly of the spring (6), followed by separation of the upper arm (19) from the lower arm (20), rotation of the lower arm (20) around its longest horizontal axis and subsequent reassembly with positioning of the mechanical component limiting the extent of movement and force (13), the spring (6) and the mechanical and pivoting component (5), and by disassembly the removable piece (15) and positioning it on the assembly surface (16) on the opposing face, thus becoming a functional system again, but now with a symmetrical position of the lower arm;
c) if the rotation is only implemented with the removable end (3), this end is disassembled by exerting a horizontal shear force, in the separation zone (22) between the cable (4) and the end (3), which will allow the sliding of the engaging V-shaped protrusion surfaces, followed by the rotation of this part along its horizontal axis, and thereafter being reassembled by pressing and sliding between the V-shaped surfaces, and posterior detachment and positioning of the component (15) for contact with the lower incisors on the assembly surface (16) of the opposing face of the removable end.

10. Mechanical device according to claim 1 wherein the two arms are articulated by the mechanical component (5) for pivoting the upper arm (19) to the lower arm (20) in which:
a) this mechanical and pivoting component (5) ensures the connection between the upper (19) and lower (20) arms, allowing an inter-arms rotational movement relatively to their axis;
b) this mechanical component (5) may be a cylindrical pin or spindle having a length which fills the interior space;
c) this mechanical and pivoting component (5) is inserted through the holes (9) of the upper arm (19) and the holes (8) of the lower arm (20) allowing the positioning of the two arms of the device, their articulation and movement.

11. Mechanical device according to claim 1 wherein it comprises a spring (6) or mechanical component between arms which:
a) engages in the notch surfaces (10) of the lower arm (20) and in the engaging and retaining area (12) of the upper arm (19);
b) may be a compression spring or other material with equivalent elastic behavior;
c) may be replaced by an active mechanical component, with ends adapted to the assembly and retention zones (12) and to the notch surfaces (10), integrating a micromotor or programmable part, allowing the linear movement to be executed in its axis with consequent separation or approximation between the handles (1) and (4).

12. Mechanical device according to claim 1 wherein it comprises a mechanical component (13) to limit the force and the extent of movement which:
a) may be fixed or movable, adjustable or not, rigid or elastic, which may have coupled digital systems or others for monitoring the force and extent of movement;
b) fixable by engagement, friction or screwing in the orifice (18) to fasten the mechanical component to limit the extent of movement and force, present on the two sides of the handle (4) of the lower arm (20) of the device;
c) the component (13) functions as a mechanical limit switch.

13. Mechanical device according to claim 1 wherein it comprises an upper arm (19) and a lower arm (20) in which:
a) their handles (1) and (4) have a smooth surface, that can be rugged or customized (for ergonomics adjusted for manual, digital gripping with left or right hand) with wavy, rounded or other shapes in order to receive the different sensors or components that may be attached to the device;
b) their handles (1) and (4) may have holes along their body for insertion of other components.

14. Mechanical device according to claim 1 wherein the lower arm (20):
a) has on its upper and lower faces of its handle (4) an engaging surface (16) for a removable piece (15) for contact with the mandibular incisors;
b) has on its top and bottom faces of its handle (4) a notch surface (10) for engaging the spring (6) or mechanical component between the upper arm (19) and the lower arm (20);
c) has in its middle zone, in the transition from the cable (4) to its end with an individualizable surface (3) with the curvature that precedes it, the hole (8) of the lower arm (20) for the mechanical component (5), for articulation and pivoting between the upper arm (19) and the lower arm (20).

15. Mechanical device according to claim 14 wherein the lower arm (20):
a) has on its upper and lower faces of its handle (4) an engaging surface (16) for a removable piece (15) for contact with the mandibular incisors;
b) has on its top and bottom faces of its handle (4) a notch surface (10) for engaging the spring (6) or mechanical component between the upper arm (19) and the lower arm (20);
c) has in its middle zone, in the transition from the cable (4) to its end with an individualizable surface (3) with the curvature that precedes it, the hole (8) of the lower arm (20) for the mechanical component (5), for articulation and pivoting between the upper arm (19) and the lower arm (20).

16. Mechanical device according to claim 8 wherein it comprises a lower arm (20) in which:
a) the rotation along the longer horizontal axis of the entire lower arm (20) or only its end (3) allows alternating the treatment of the left or right TMJ, using only the same device and not two different devices, depending on the indications or needs of the user;
b) in the case of rotation of the entire lower arm (20) this is achieved by the temporary removal of the mechanical and pivoting component (5), removal of the mechanical component to limit the extent of movement and force (13), disassembly of the spring (6), followed by separation of the upper arm (19) from the lower arm (20), rotation of the lower arm (20) around its longest horizontal axis and subsequent reassembly with positioning of the mechanical component limiting the extent of movement and force (13), the spring (6) and the mechanical and pivoting component (5), and by disassembly the removable piece (15) and positioning it on the assembly surface (16) on the opposing face, thus becoming a functional system again, but now with a symmetrical position of the lower arm;
c) if the rotation is only implemented with the removable end (3), this end is disassembled by exerting a horizontal shear force, in the separation zone (22) between the cable (4) and the end (3), which will allow the sliding of the engaging V-shaped protrusion surfaces, followed by the rotation of this part along its horizontal axis, and thereafter being reassembled by pressing and sliding between the V-shaped surfaces, and posterior detachment and positioning of the component (15) for contact with the lower incisors on the assembly surface (16) of the opposing face of the removable end.

17. Mechanical device according to claim 1 wherein it comprises a removable piece (15) for contact with the mandibular incisors, which fits in the engaging surface (16) of the handle (4) of the lower arm (20).

18. Mechanical device according to claim 15 wherein it comprises a removable piece (15) for contact with the mandibular incisors, which fits in the engaging surface (16) of the handle (4) of the lower arm (20).

19. Mechanical device according to claim 16 wherein it comprises a removable piece (15) for contact with the mandibular incisors, which fits in the engaging surface (16) of the handle (4) of the lower arm (20).
